SEQUENCE LISTING (67 sequences)

SEQ ID NO: 1 - DNA, 174 bp, Homo sapiens
ccagggttcttgtgaagatcaagtgataatgtaggaagcatgtacaaatgtcacattctgccgtcacgtaatggtcctcacagcttgaggtagcatttagcatgtgtcatgatttagtacaagggttggcaaactgttgctcttggattaagtctggctcattgcctgtttttc

SEQ ID NO: 2 - DNA, 374 bp, Homo sapiens
caggtccttggatttctttctgtgaaaatgaaagcataggtaggaatttcccatggaacagctagcagaggagaaatattaaaagtcaggagactcatgctatagttttcatacttcattacaacaatgttgtttaggacaagtgagttaacctgttagcttcctctatataaaatggaaagtcattaaaaacctacatagcagggttcttgtgaagatcaagtgataatgtaggaagcatgtacaaatgtcacattctgccgtcacgtaatggtcctcacagcttgaggtagcatttagcatgtgtcatgatttagtacaagggttggcaaactgttgctcttggattaagtctggctcattgcctgtttttc

SEQ ID NO: 3 - DNA, 375 bp, Homo sapiens
  CDS (1)..(375): RF +1
caggtccttggatttctttctgtgaaaatgaaagcataggtaggaatttcccatggaacagctagcagaggagaaatattaaaagtcaggagactcatgctatagttttcatacttcattacaacaatgttgtttaggacaagtgagttaacctgttagcttcctctatataaaatggaaagtcattaaaaacctacatagcagggttcttgtgaagatcaagtgataatgtaggaagcatgtacaaatgtcacattctgccgtcacgtaatggtcctcacagcttgaggtagcatttagcatgtgtcatgatttagtacaagggttggcaaactgttgctcttggattaagtctggctcattgcctgtttttcn

SEQ ID NO: 4 - DNA, 374 bp, Homo sapiens; identical to SEQ ID NO: 2
  CDS (2)..(373)
  RF +2

SEQ ID NO: 5 - DNA, 374 bp, Homo sapiens; identical to SEQ ID NO: 2
  CDS (3)..(374)
  RF +3

SEQ ID NO: 6 - DNA, 374 bp, Homo sapiens
  RF -1
gaaaaacaggcaatgagccagacttaatccaagagcaacagtttgccaacccttgtactaaatcatgacacatgctaaatgctacctcaagctgtgaggaccattacgtgacggcagaatgtgacatttgtacatgcttcctacattatcacttgatcttcacaagaaccctgctatgtaggtttttaatgactttccattttatatagaggaagctaacaggttaactcacttgtcctaaacaacattgttgtaatgaagtatgaaaactatagcatgagtctcctgacttttaatatttctcctctgctagctgttccatgggaaattcctacctatgctttcattttcacagaaagaaatccaaggacctg

SEQ ID NO: 7 - DNA, 374 bp, Homo sapiens; identical to SEQ ID NO: 6
  RF -2

SEQ ID NO: 8 - DNA, 374 bp, Homo sapiens; identical to SEQ ID NO: 6
  RF -3

SEQ ID NO: 9 - protein, 45 aa, Homo sapiens
MLNATSSCEDHYVTAECDICTCFLHYHLIFTRTLLCRFLMTFHFI

SEQ ID NO: 10 - DNA, 221 bp, Homo sapiens
acaggaaggacggtcagagatgattccaacatgaggactttgctagttgcagtgtctgactcctcccattctcatagcagttttgcacagtagttttggtttgtgtgtatgttttaggcaatgtgacaaagcagttttcctcctgcctccttgagaaatttaatcctgacaggttctttaagaaatcagcttgatgtgggctgattttaaaagaataaatg

SEQ ID NO: 11 - DNA, 621 bp, Homo sapiens
agggacgtatcactgtgtcttctcaaaatggtgggtgtgaatggaatttttcaaagttgaatcaatttgagatatttctgaagtttgtttaggaaacaagtatgtgactaccttcctaagagctgactggttttttgaggcctggattgtgtgtgtatgtgcttatgtgtgtgttataaaatggaatcaactataccaggttaaacaaatggacaagtcatggaaaagacacagctctgtttggacattaataagggcgggtgtggaagctggaaagctgagatgccggaaggctcagtgcttagtaggcccggaaaagcaatttttgaactggcatctcattccccttttctaagacaggaaggacggtcagagatgattccaacatgaggactttgctagttgcagtgtctgactcctcccattctcatagcagttttgcacagtagttttggtttgtgtgtatgttttaggcaatgtgacaaagcagttttcctcctgcctccttgagaaatttaatcctgacaggttctttaagaaatcagcttgatgtgggctgattttaaaagaataaatgaaaaaaaaaagctggtgactgatagctgttttattggtcttgta

SEQ ID NO: 12 - protein, 56 aa, Homo sapiens [sequence omitted]

SEQ ID NO: 13 - DNA, 356 bp, Homo sapiens
caagcacaaagagcttgctccctacgatgagaactggttctacacgcgagctgcttccacagcgcggcacctgtacctccggggtggcgctggggttggctccatgaccaagatctatgggggacgtcagagaaacggcgtcatgcccagccacttcagccgaggctccaagagtgtggcccgccgggtcctccaagccctggaggggctgaaaatggtggaaaaggaccaagatggcggccgcaaactgacacctcagggacaaagagatctggacagaatcgccggacaggtggcagctgccaacaagaagcattagaacaaaccatgctgggttaataaattgcctcattcgt

SEQ ID NO: 14 - DNA, 873 bp, Homo sapiens
  CDS (373)..(810)
gtactttcgccatcatagtattctccaccactgttccttccagccacgaacgacgcaaacgaagccaagttcccccagctccgaacaggagctctctatcctctctctattacactccgggagaaggaaacgcgggaggaaacccaggcctccacgcgcgaccccttggccctcccctttacctctccacccctcactagacaccctcccctctaggcggggacgaactttcgccctgagagaggcggagcctcagcgtctaccctcgctctcgcgagctttcggaactctcgcgagaccctacgcccgacttgtgcgcccgggaaaccccgtcgttccctttcccctggctggcagcgcggaggccgcacgatgcctggagttactgtaaaagacgtgaaccagcaggagttcgtcagagctctggcagccttcctcaaaaagtccgggaagctgaaagtccccgaatgggtggataccgtcaagctggccaagcacaaagagcttgctccctacgatgagaactggttctacacgcgagctgcttccacagcgcggcacctgtacctccggggtggcgctggggttggctccatgaccaagatctatgggggacgtcagagaaacggcgtcatgcccagccacttcagccgaggctccaagagtgtggcccgccgggtcctccaagccctggaggggctgaaaatggtggaaaaggaccaagatggcggccgcaaactgacacctcagggacaaagagatctggacagaatcgccggacaggtggcagctgccaacaagaagcattagaacaaaccatgctgggttaataaattgcctcattcgtaaaaaaaaaaaaaaaaaaaaaaaaaa

SEQ ID NO: 15 - protein, 145 aa, Homo sapiens [sequence omitted]

SEQ ID NO: 16 - protein, 12 aa, Homo sapiens
  Peptide 1 encoded by RF +1 of SEQ ID NO 2
QVLGFLSVKMKA

SEQ ID NO: 17 - protein, 21 aa, Homo sapiens
  Peptide 2 encoded by RF +1 of SEQ ID NO 2
VGISHGTASRGEILKVRRLML

SEQ ID NO: 18 - protein, 17 aa, Homo sapiens
  Peptide 3 encoded by RF +1 of SEQ ID NO 2
FSYFITTMLFRTSELTC

SEQ ID NO: 19 - protein, 9 aa, Homo sapiens
  Peptide 4 encoded by RF +1 of SEQ ID NO 2
LPLYKMESH

SEQ ID NO: 20 - protein, 3 aa, Homo sapiens
  Peptide 5 encoded by RF +1 of SEQ ID NO 2
KPT

SEQ ID NO: 21 - protein, 10 aa, Homo sapiens
  Peptide 6 encoded by RF +1 of SEQ ID NO 2
QGSCEDQVIM

SEQ ID NO: 22 - protein, 17 aa, Homo sapiens
  Peptide 7 encoded by RF +1 of SEQ ID NO 2
EACTNVTFCRHVMVLTA

SEQ ID NO: 23 - protein, 3 aa, Homo sapiens
  Peptide 8 encoded by RF +1 of SEQ ID NO 2
GSI

SEQ ID NO: 24 - protein, 3 aa, Homo sapiens
  Peptide 9 encoded by RF +1 of SEQ ID NO 2
HVS

SEQ ID NO: 25 - protein, 12 aa, Homo sapiens
  Peptide 10 encoded by RF +1 of SEQ ID NO 2
FSTRVGKLLLLD

SEQ ID NO: 26 - protein, 8 aa, Homo sapiens
  Peptide 11 encoded by RF +1 of SEQ ID NO 2
VWLIACFS

SEQ ID NO: 27 - protein, 7 aa, Homo sapiens
  Peptide 1 encoded by RF +2 of SEQ ID NO 2
RSLDFFL

SEQ ID NO: 28 - protein, 1 aa, Homo sapiens
  Peptide 2 encoded by RF +2 of SEQ ID NO 2
K

SEQ ID NO: 29 - protein, 3 aa, Homo sapiens
  Peptide 3 encoded by RF +2 of SEQ ID NO 2
KHR

SEQ ID NO: 30 - protein, 12 aa, Homo sapiens
  Peptide 4 encoded by RF +2 of SEQ ID NO 2
EFPMEQLAEEKY

SEQ ID NO: 31 - protein, 22 aa, Homo sapiens
  Peptide 5 encoded by RF +2 of SEQ ID NO 2
KSGDSCYSFHTSLQQCCLGQVS

SEQ ID NO: 32 - protein, 24 aa, Homo sapiens
  Peptide 6 encoded by RF +2 of SEQ ID NO 2
PVSFLYIKWKVIKNLHSRVLVKIK

SEQ ID NO: 33 - protein, 13 aa, Homo sapiens
  Peptide 7 encoded by RF +2 of SEQ ID NO 2
CRKHVQMSHSAVT

SEQ ID NO: 34 - protein, 34 aa, Homo sapiens
  Peptide 8 encoded by RF +2 of SEQ ID NO 2
WSSQLEVAFSMCHDLVQGLANCCSWIKSGSLPVF

SEQ ID NO: 35 - protein, 20 aa, Homo sapiens
  Peptide 1 encoded by RF +3 of SEQ ID NO 2
GPWISFCENESIGRNFPWNS

SEQ ID NO: 36 - protein, 23 aa, Homo sapiens
  Peptide 2 encoded by RF +3 of SEQ ID NO 2
QRRNIKSQETHAIVFILHYNNVV

SEQ ID NO: 37 - protein, 2 aa, Homo sapiens
  Peptide 3 encoded by RF +3 of SEQ ID NO 2
DK

SEQ ID NO: 38 - protein, 8 aa, Homo sapiens
  Peptide 4 encoded by RF +3 of SEQ ID NO 2
VNLLASSI

SEQ ID NO: 39 - protein, 13 aa, Homo sapiens
  Peptide 5 encoded by RF +3 of SEQ ID NO 2
NGKSLKTYIAGFL

SEQ ID NO: 40 - protein, 25 aa, Homo sapiens
  Peptide 6 encoded by RF +3 of SEQ ID NO 2
RSSDNVGSMYKCHILPSRNGPHSLR

SEQ ID NO: 41 - protein, 7 aa, Homo sapiens
  Peptide 7 encoded by RF +3 of SEQ ID NO 2
HLACVMI

SEQ ID NO: 42 - protein, 19 aa, Homo sapiens
  Peptide 8 encoded by RF +3 of SEQ ID NO 2
YKGWQTVALGLSLAHCLFF

SEQ ID NO: 43 - protein, 8 aa, Homo sapiens
  Peptide 1 encoded by RF -1 of SEQ ID NO 2
EKQAMSQT

SEQ ID NO: 44 - protein, 16 aa, Homo sapiens
  Peptide 2 encoded by RF -1 of SEQ ID NO 2
SKSNSLPTLVLNHDTC

SEQ ID NO: 45 - protein, 10 aa, Homo sapiens
  Peptide 3 encoded by RF -1 of SEQ ID NO 2
MLPQAVRTIT

SEQ ID NO: 46 - protein, 14 aa, Homo sapiens
  Peptide 4 encoded by RF -1 of SEQ ID NO 2
RQNVTFVHASYIIT

SEQ ID NO: 47 - protein, 10 aa, Homo sapiens
  Peptide 5 encoded by RF -1 of SEQ ID NO 2
SSQEPCYVGF

SEQ ID NO: 48 - protein, 8 aa, Homo sapiens
  Peptide 6 encoded by RF -1 of SEQ ID NO 2
LSILYRGS

SEQ ID NO: 49 - protein, 51 aa, Homo sapiens
  Peptide 7 encoded by RF -1 of SEQ ID NO 2
QVNSLVLNNIVVMKYENYSMSLLTFNISPLLAVPWEIPTYAFIFTERNPRT

SEQ ID NO: 50 - protein, 4 aa, Homo sapiens
  Peptide 1 encoded by RF -2 of SEQ ID NO 2
KNRQ

SEQ ID NO: 51 - protein, 14 aa, Homo sapiens
  Peptide 2 encoded by RF -2 of SEQ ID NO 2
ARLNPRATVCQPLY

SEQ ID NO: 52 - protein, 11 aa, Homo sapiens
  Peptide 3 encoded by RF -2 of SEQ ID NO 2
IMTHAKCYLKL

SEQ ID NO: 53 - protein, 8 aa, Homo sapiens
  Peptide 4 encoded by RF -2 of SEQ ID NO 2
GPLRDGRM

SEQ ID NO: 54 - protein, 18 aa, Homo sapiens
  Peptide 5 encoded by RF -2 of SEQ ID NO 2
HLYMLPTLSLDLHKNPAM

SEQ ID NO: 55 - protein, 19 aa, Homo sapiens
  Peptide 6 encoded by RF -2 of SEQ ID NO 2
VFNDFPFYIEEANRLTHLS

SEQ ID NO: 56 - protein, 4 aa, Homo sapiens
  Peptide 7 encoded by RF -2 of SEQ ID NO 2
TTLL

SEQ ID NO: 57 - protein, 6 aa, Homo sapiens
  Peptide 8 encoded by RF -2 of SEQ ID NO 2
SMKTIA

SEQ ID NO: 58 - protein, 2 aa, Homo sapiens
  Peptide 9 encoded by RF -2 of SEQ ID NO 2
VS

SEQ ID NO: 59 - protein, 7 aa, Homo sapiens
  Peptide 10 encoded by RF -2 of SEQ ID NO 2
LLIFLLC

SEQ ID NO: 60 - protein, 20 aa, Homo sapiens
  Peptide 11 encoded by RF -2 of SEQ ID NO 2
LFHGKFLPMLSFSQKEIQGP

SEQ ID NO: 61 - protein, 21 aa, Homo sapiens
  Peptide 1 encoded by RF -3 of SEQ ID NO 2
KTGNEPDLIQEQQFANPCTKS

SEQ ID NO: 62 - protein, 46 aa, Homo sapiens
  Peptide 2 encoded by RF -3 of SEQ ID NO 2
HMLNATSSCEDHYVTAECDICTCFLHYHLIFTRTLLCRFLMTFHFI

SEQ ID NO: 63 - protein, 5 aa, Homo sapiens
  Peptide 3 encoded by RF -3 of SEQ ID NO 2
RKLTG

SEQ ID NO: 64 - protein, 12 aa, Homo sapiens
  Peptide 4 encoded by RF -3 of SEQ ID NO 2
LTCPKQHCCNEV

SEQ ID NO: 65 - protein, 2 aa, Homo sapiens
  Peptide 5 encoded by RF -3 of SEQ ID NO 2
KL

SEQ ID NO: 66 - protein, 6 aa, Homo sapiens
  Peptide 6 encoded by RF -3 of SEQ ID NO 2
HESPDF

SEQ ID NO: 67 - protein, 26 aa, Homo sapiens
  Peptide 7 encoded by RF -3 of SEQ ID NO 2
YFSSASCSMGNSYLCFHFHRKKSKDL

(19) United States
(12) Patent Application Publication
(10) Pub. No.: US 2010/0047770 A1
(43) Pub. Date: Feb. 25, 2010

(54) Detection of Breast Cancer

(75) Inventors: Martin Andrew Crockard, Northern Ireland (GB); John Victor Lamont, Northern Ireland (GB); Stephen Peter Fitzgerald, Northern Ireland (GB)

Correspondence Address: MARSHALL, GERSTEIN & BORUN LLP, 233 SOUTH WACKER DRIVE, 6300 SEARS TOWER, CHICAGO, IL, 60606-6357, US

(73) Assignee: RANDOX LABORATORIES LTD., Crumlin. Co. Antrim, Northern Ireland (GB)

(21) Appl. No.: 11/576,521

(22) PCT Filed: Oct. 6, 2005
(86) PCT No.: PCT/GB05/03841

§ 371 Date: Jan. 7, 2008

(30) Foreign Application Priority Data
Oct. 6, 2004 (GB) 0422211.3

(51) Int. Cl. C12Q 1/68; C07K 7/08; C07K 14/435; C07K 16/00; C07H 21/04

(52) U.S. Cl. 435/6; 530/324; 530/325; 530/326; 530/350; 530/387.9; 536/23.1; 536/24.5

(57) ABSTRACT

A method for the detection of the presence or risk of cancer in a patient, comprises the steps of: (i) isolating a sample of the patient's genome; and (ii) detecting the presence or expression of the gene characterized by any of the nucleotide sequences identified as SEQ ID No 1, SEQ ID No. 10 and SEQ ID No. 13 wherein the presence or expression of the gene indicates the presence of or the risk of cancer.

### FIELD OF THE INVENTION

This invention relates to the detection of the presence of, or the risk of cancer, in particular, breast cancer.

### BACKGROUND OF THE INVENTION

There are over 1 million cases of breast cancer per year on a global basis, of which around 0.5 million are in the US, 40,000 are in the UK and nearly 2,000 in Ireland. It is the leading cause of cancer deaths among women (Keen and Davidson, 2003). Although the overall incidence of the disease is increasing within the western world, wider screening and improved treatments have led to a gradual decline in the fatality rate of about 1% per year since 1991. Inheritance of susceptibility genes, such as BRCA1 and BRCA2, account for only 5% of breast cancer cases and the factors responsible for the other 95% remain obscure (Grover and Martin, 2002). In the absence of a strategy to reduce causative agents of breast cancer, early detection remains the best approach to reducing the mortality rate of this disease. It is widely held that breast cancer initiates as the pre-malignant stage of atypical ductal hyperplasia (ADH), progresses into the pre-invasive stage of ductal carcinoma in situ (DCIS), and culminates in the potentially lethal stage of invasive ductal carcinoma (IDC). This linear model of breast cancer progression has been the rationale for the use of detection methods such as mammography in the hope of diagnosing and treating breast cancer at earlier clinical stages (Ma et al., 2003).

Patients diagnosed with early breast cancer have greater than a 90% 5 year relative survival rate, as compared to 20% for patients diagnosed with distally metastasised breast cancer. Nonetheless, there is no definitive early-stage screening test for breast cancer, diagnosis currently being made on the results of mammography and fine needle biopsy. Mammography has its limitations, with over 80% of suspicious results being false positives and 10-15% of women with breast cancer providing false negative results. Often the tumour has reached a late stage in development before detection, reducing the chances of survival for the patient and increasing the cost of treatment and management for the healthcare system. More sensitive methods are required to detect small (<2 cm diameter) early stage in-situ carcinomas of the breast, to reduce patient mortality. In addition to early detection, there remain serious problems in classifying the disease as malignant or benign, in the staging of known cancers and in differentiating between tumour types. Finally, there is a need to monitor ongoing treatment effects and to identify patients becoming resistant to particular therapies. Such detection processes are further complicated, as the mammary gland is one of the few organs that undergo striking morphological and functional changes during adult life, particularly during pregnancy, lactation and involution, potentially leading to changes in the molecular signature of the same mammary gland over time.

Diagnosis of disease is often made by the careful examination of the relative levels of a small number of biological markers. Despite recent advances, the contribution of the current biomarkers to patient care and clinical outcome is limited. This is due to their low diagnostic sensitivity and disease specificity. Some molecular biomarkers, however, are being used routinely in disease diagnosis, for example prostate specific antigen in prostate cancer screening, and new candidate markers are being discovered at an increasing rate (Pritzker, 2002). It is becoming accepted that the use of a panel of well-validated biomarkers would enhance the positive predictive value of a test and minimize false positives or false negatives (Srinivas et al., 2002). In addition, there is now growing interest in neural networks, which show the promise of combining weak but independent information from various biomarkers to produce a prognostic/predictive index that is more informative than each biomarker alone (Yousef et al., 2002).

As more molecular information is being collated, diseases such as breast cancer are being sub-divided according to genetic signatures linked to patient outcome, providing valuable information for the clinician. Emerging novel technologies in molecular medicine have already demonstrated their power in discriminating between disease sub-types that are not recognisable by traditional pathological criteria (Sorlie et al., 2001) and in identifying specific genetic events involved in cancer progression (Srinivas et al., 2002). Further issues need to be addressed in parallel, relating to the efficacy of biomarkers between genders and races, thus large scale screening of a diverse-population is a necessity.

In addition, the management of breast cancer could be improved by the use of new markers normally expressed only in the breast but found elsewhere in the body, as a result of the disease. Predictors of the activity of the disease would also have valuable utility in the management of the disease, especially those that predict if a ductal carcinoma in situ will develop into invasive ductal carcinoma.

### SUMMARY OF THE INVENTION

According to a first aspect of the present invention, there is a method for the detection of the presence of or the risk of cancer in a patient, comprising the steps of:
(i) isolating a sample of the patient's genome; and
(ii) detecting the presence or expression of any of the genes characterised by the nucleotide sequences identified as SEQ ID No. 1, SEQ ID No.10 and SEQ ID No.13, wherein the presence or expression of the genes indicates the presence of or the risk of cancer.

According to a second aspect of the invention, an isolated polynucleotide comprises any of the nucleotide sequences identified herein as SEQ ID No 1, SEQ ID No. 10 or SEQ ID No.13, or their complements, or a polynucleotide of at least 15 consecutive nucleotides that hybridises to any of the sequences (or their complements) under stringent hybridising conditions.

According to a third aspect of the present invention, an isolated peptide comprises any of the sequences identified herein as SEQ ID No. 9, SEQ ID No. 12 and SEQ ID No. 15, or a fragments thereof of at least 10 consecutive amino acid residues.

According to a fourth aspect of the present invention, an antibody has an affinity of at least 10⁻⁶M for a peptide as defined above.

According to a fifth aspect of the invention, a polynucleotide that hybridises to or otherwise inhibits the expression of an endogenous DD53 gene, is used in the manufacture of a medicament for the treatment of cancer, in particular breast cancer.

According to a sixth aspect of the invention, a polynucleotide that hybridises to or otherwise inhibits the expression of an endogenous TS32 gene, is used in the manufacture of a medicament for the treatment of cancer, in particular breast cancer.

According to a seventh aspect of the invention, a polynucleotide that hybridises to or otherwise inhibits the expression of an endogenous IDC25 gene, is used in the manufacture of a medicament for the treatment of cancer, in particular breast cancer.

### DESCRIPTION OF THE INVENTION

The present invention is based on the identification of genes that are expressed in a patient suffering cancer, in particular, breast cancer. Identification of the individual genes (or their expressed products) in a sample obtained from a patient indicates the presence of or the risk of cancer in the patient.

The invention further relates to reagents such as polypeptide sequences, useful for detecting, diagnosing, monitoring, prognosticating, preventing, imaging, treating or determining a pre-disposition to cancer.

The methods to carry out the diagnosis can involve the synthesis of cDNA from the mRNA in a test sample, amplifying as appropriate portions of the cDNA corresponding to the genes or fragments thereof and detecting each product as an indication of the presence of the disease in that tissue, or detecting translation products of the mRNAs comprising gene sequences as an indication of the presence of the disease.

Useful reagents include polypeptides or fragment(s) thereof which may be useful in diagnostic methods such as RT-PCR, PCR or hybridisation assays of mRNA extracted from biopsied tissue, blood or other test samples; or proteins which are the translation products of such mRNAs; or antibodies directed against these proteins. These assays also include methods for detecting the gene products (proteins) in light of possible post-translation modifications that can occur in the body, including interactions with molecules such as co-factors, inhibitors, activators and other proteins in the formation of sub-unit complexes.

Diagnosis can be made on the basis of the presence or absence of the gene product or by measuring increased expression of the gene in the patient.

The genes associated with cancer, are characterised by the polynucleotides shown as SEQ ID No. 1, SEQ ID No. 10 and SEQ ID No. 13. The expressed products of the genes are identified herein by SEQ ID No. 9, SEQ ID No. 12 and SEQ ID No. 15, respectively. Identification of the genes or their expressed products may be carried out by conventional techniques known for the detection or characterisation of polynucleotides or polypeptides. For example, isolated genetic material from a patient can be probed using short oligonucleotides that hybridise specifically to the target gene. The oligonucleotide probes may be detectably labelled, for example with a fluorophore, so that upon hybridisation with the target gene, the probes can be detected. Alternatively, the gene, or parts thereof, may be amplified using the polymerase chain reaction, with the products being identified, again using labelled oligonucleotides.

Diagnostic assays incorporating any of these genes, proteins or antibodies will include, but not be limited to:

Polymerase chain reaction (PCR)

Reverse transcription PCR

Real-time PCR

In-situ hybridisation

Southern dot blots

Immuno-histochemistry

Ribonuclease protection assay

cDNA array techniques

ELISA

Protein, antigen or antibody arrays on solid supports such as glass or ceramics.

Small interfering RNA functional assays.

All of the above techniques are well known to those in the art.

The present invention is also concerned with isolated polynucleotides that comprise the sequences identified as SEQ ID No. 1, SEQ ID No. 10 and SEQ ID No. 13, or their complements, or fragments thereof that comprise at least 15 consecutive nucleotides, preferably 30 nucleotides, more preferably at least 50 nucleotides. Polynucleotides that hybridise to a polynucleotide as defined above, are also within the scope of the invention. Hybridisation will usually be carried out under stringent conditions, known to those in the art and are chosen to reduce the possibility of non-complementary hybridisation. Examples of suitable conditions are disclosed in Nucleic Acid Hybridisation. A Practical Approach (B. D. Hames and S. J. Higgins, editors IRL Press, 1985).

The identification of the DD53 gene (SEQ ID No. 1), the TS32 gene (SEQ ID No. 10) and the IDC25 gene (SEQ ID No. 13) also permits therapies to be developed, with each gene being a target for therapeutic molecules. For example, there are now many known molecules that have been developed for gene therapy, to target and prevent the expression of a specific gene. One particular molecule is a small interfering RNA (siRNA), which suppresses the expression of a specific target protein by stimulating the degradation of the target mRNA. Other synthetic oligonucleotides are also known which can bind to a gene of interest (or its regulatory elements) to modify expression. Peptide nucleic acids (PNAs) in association with DNA (PNA-DNA chimeras) have also been shown to exhibit strong decoy activity, to alter the expression of the gene of interest.

The present invention also includes antibodies raised against a peptide expressed by any of the genes identified in the invention. The antibodies will usually have an affinity for the peptide of at least 10⁻⁶M, more preferably, 10⁻⁹M and most preferably at least 10⁻¹¹M. The antibody may be of any suitable type, including monoclonal or polyclonal. Assay kits for determining the presence of the peptide antigen in a test sample are also included. In one embodiment, the assay kit comprises a container with an antibody, which specifically binds to the antigen, wherein the antigen comprises at least one epitope encoded by the DD53 gene, the TS32 gene or the IDC25 gene. These kits can further comprise containers with useful tools for collecting test samples, such as blood, saliva, urine and stool. Such tools include lancets and absorbent paper or cloth for collecting and stabilising blood, swabs for collecting and stabilising saliva, cups for collecting and stabilising urine and stool samples. The antibody can be attached to a solid phase, such as glass or a ceramic surface.

Detection of antibodies that specifically bind to any of the antigens in a test sample suspected of containing these antibodies may also be carried out. This detection method comprises contacting the test sample with a polypeptide, which contains at least one epitope of the gene. Contacting is performed for a time and under conditions sufficient to allow antigen/antibody complexes to form. The method further entails detecting complexes, which contain any of the polypeptides. The polypeptide complex can be produced recombinantly or synthetically or be purified from natural sources.

In a separate embodiment of the invention, antibodies, or fragments thereof, against any of the antigens (peptides) can be used for the detection of image localisation of the antigen in a patient for the purpose of detecting or diagnosing the disease or condition. Such antibodies can be monoclonal or polyclonal, or made by molecular biology techniques and can be labelled with a variety of detectable agents, including, but not limited to radioisotopes.

In a further embodiment of the invention, antibodies or fragments thereof, whether monoclonal or polyclonal or made by molecular biology techniques, can be used as therapeutics for the disease characterised by the expression of any of the genes of the invention. The antibody may be used without derivatisation, or it may be derivatised with a cytotoxic agent such as radioisotope, enzyme, toxin, drug, pro-drug or the like.

The term “antibody” refers broadly to any immunologic binding agent such as IgG, IgM, IgA, IgD and IgE. Antibody is also used to refer to any antibody-like molecule that has an antigen-binding region and includes antibody fragments such as single domain antibodies (DABS), Fv, scFv, aptamers, etc. The techniques for preparing and using various antibody-based constructs and fragments are well known in the art. Means for preparing and characterising antibodies are also well known in the art.

If desired, the cancer screening methods of the present invention may be readily combined with other methods in order to provide an even more reliable indication of diagnosis or prognosis, thus providing a multi-marker test.

The following examples illustrate the invention with reference to the accompanying drawings.

### EXAMPLES

### DD53, TS32 and IDC25

A number of differentially expressed gene fragments were isolated from cDNA populations derived from matched clinical samples of breast cancer patients, using non-isotopic differential display (DDRT-PCR). Three of these fragments, DD53, TS32 and IDC25, showed a significant increase in expression in breast tumour tissue samples from a number of donors, in comparison to their co-excised normal tissue counterparts. The expression profile of these novel molecular markers, their full length and corresponding presumed protein sequences are detailed herein.

### Materials and Methods

Differential gene expression was investigated between matched pairs of normal mammary and tumour tissue from the same donor. Tissue samples were obtained, with full ethical approval and informed patient consent, from Medical Solutions plc, Nottingham, UK. Following the surgical removal of a tumour, one sample of the tumour tissue was collected, as was a sample from the adjacent, co-excised normal tissue. Messenger RNA was extracted and cDNA subsequently synthesised, using Dynal dT₁₈-tagged Dynabeads and Superscript II reverse transcription protocols, respectively. Differential display reverse transcription PCR (DDRT-PCR) was employed to observe differences between the gene expression profiles of these matched samples and individual gene transcripts showing up- or down-regulation were isolated and investigated further.

First described by Liang & Pardee (1992) differential display reverse transcription PCR (DDRT-PCR) uses mRNA from two or more biological samples as templates for representative cDNA synthesis by reverse transcription, with one of 3 possible anchor primers. Each of the 3 sub-populations was PCR amplified using its respective anchor primer coupled with one of 80 arbitrary 13-mer primers. This number of primer combinations has been estimated to facilitate the representation of 96% of expressed genes in an mRNA population (Sturtevant, 2000). This population sub-division results in the reduction of the estimated 12,000-15,000 mRNAs expressed in eukaryotic cells to 100-150 transcripts on completion of second strand cDNA synthesis for each primer set. This facilitates the parallel electrophoretic separation and accurate visualization of matched primer sets on a polyacrylamide gel, leading to the identification of gene fragments expressed in one tissue sample but not the other.

Excision and re-amplification of fragments of interest was followed by removal of false positives through reverse Southern dot blotting. This entailed the spotting of each re-amplified fragment onto duplicate nylon membranes (Hybond N+, Amersham Pharmacia Biotech) and hybridising these with either the tumour or normal tissue cDNA population of the donor from which the fragments were derived. Those fragments confirmed as differentially expressed were then cloned and sequenced, followed by web-based database interrogation to determine if each gene was novel and to find its chromosomal location. Fragments not matching known genes were regarded as potentially representing novel markers for the breast cancer from which they were derived. Further screening of each transcript was performed by either semi-quantitative RT-PCR or real-time PCR, using a suite of matched cDNA populations from a number of breast tumour donors. In all cases, M-actin was used as a constitutive reference gene, for calibrating the cDNA templates and as an internal positive control during PCR. Expression of each putative novel marker gene was performed by PCR using gene-specific primer sets on the calibrated matched templates. Potential full-length transcripts of the novel gene fragments, including the open reading frame (that piece of the gene that encodes the protein) were then synthesized using 5′ RACE (rapid amplification of cDNA ends), which incorporates gene-specific extension and amplification, verifiable by sequencing. Alternatively, homologous known sequences to the putative markers were exploited, such as in the case of IDC25 and its homologue S19, with primers being designed for their corresponding open reading frames, followed by sequence verification of the amplified products.

The tissue specific expression profile of each molecular marker was tested using gene specific primers against cDNA populations derived from a comprehensive panel of 22 human tissue types. These are as follows:

| | |
|---|---|
| Adrenal gland | pooled from 62 donors |
| Bone marrow | pooled from 7 donors |
| Brain, cerebellum | pooled from 24 donors |
| Brain, whole | pooled from one donor |
| Colon* | pooled from one donor |
| Foetal brain | pooled from 59 donors |
| Foetal liver | pooled from 63 donors |
| Heart | pooled from one donor |
| Kidney | pooled from one donor |
| Liver | pooled from one donor |
| Lung | pooled from one donor |
| Placenta | pooled from 7 donors |
| Prostate | pooled from 47 donors |
| Salivary gland | pooled from 24 donors |
| Skeletal muscle | pooled from 2 donors |
| Small intestine* | pooled from one donor |
| Spleen | pooled from 14 donors |
| Testis | pooled from 19 donors |
| Thymus | pooled from 9 donors |
| Thyroid gland | pooled from 65 donors |
| Trachea | pooled from 1 donor |
| Uterus | pooled from 10 donors |

The majority of these samples were part of the Human Total RNA panel 11 (Clontech), but two RNA samples, marked with asterisks, were obtained separately from Clontech. In addition, assays were performed on a range of ethically approved human tumour samples, obtained through Medical Solutions plc, Nottingham, UK. DNA representative of tumours from ovary, testis, stomach, liver, lung, bladder, colon and pancreas was tested against both β-actin and the putative markers, by real-time and conventional PCR.

In conjunction with novel marker expression analysis, each matched pair of breast tissues was subjected to molecular signature analysis. This entailed using of a suite of primers specific to a number of pre-published breast cancer molecular markers in semi-quantitative RT-PCR against each tissue cDNA. The relationship between each molecular marker was determined, tabulated for each sample and used as a reference, against which the novel markers could be compared. This is with the aim of sub-classifying the tumour types and enabling the association of novel markers against such sub-types, increasing the power of the diagnostic marker considerably.

Using differential display, a gene fragment, DD53, derived from cDNA populations of matched tissue from a breast cancer donor, was observed to have significant up-regulation in the tumour cDNA population in comparison to the corresponding normal tissue cDNA. This 174-nucleotide product (SEQ ID No. 1) was confirmed as differentially expressed by reverse Southern dot blots. Sequence analysis followed by database interrogation determined that DD53 was not homologous to known genes or proteins in the EMBL and SWISSPROT databases, respectively, so was regarded as potentially novel. It was, however, 100% homologous, after removal of the poly-A tail, to a region on chromosome 8q of the human genome.

A detailed real-time expression profile of this fragment was undertaken using cDNA populations derived from a number of matched breast and breast tumour tissue samples donated by other patients. Of the donor samples screened, many exhibited notable increases in expression, suggesting DD53 may be a putative molecular marker for the presence of breast tumour. This is represented in the table below.

| | |
|---|---|
| 2 fold | 4 fold |
| difference | difference |

| | | | | |
|---|---|---|---|---|
| DD53 Increased in tumour | 8 | 22% | 3 | 8% |
| DD53 Increased in normal | 12 | 32% | 8 | 22% |
| DD53 No discernable difference | 14 | 38% | 23 | 62% |
| DD53 No expression evident | 3 | 8% | 3 | 8% |
| Totals | 37 | 100% | 37 | 100 |

To facilitate further analysis, 5′-RACE was employed to extend the fragment to include the full open reading frame (ORF) of the gene, plus the 5′ non-coding sequence. Using this technique, a presumed full-length product of 385 nucleotides was derived (374 nucleotides without the poly A tail), which on subsequent database interrogation, confirmed the previous homology to human chromosome 8, being 100% homologous over the full length of the sequence, without the poly-A tail (SEQ ID No 2). From this sequence, all 6 amino acid reading frames were generated (SEQ ID Nos 3 to 8) and a putative, small ORF was found in the −1 frame, comprising 45 amino acids (SEQ ID No 9). This small protein failed to reveal a high homology to any known proteins in the SWALL database, so is assumed to be novel.

To determine organ specificity, cDNA populations from 8 non-breast human tissues (purchased from Origene) were tested against the DD53 primers, in addition to a matched pair of cDNAs from a breast cancer donor. The same samples were also tested using primers from the constitutive housekeeping gene, β-actin, as a positive control and to calibrate the templates for semi-quantitative PCR analysis. The β-actin product was strongly amplified in all cDNA populations studied, whereas the DD53 product was only detected in the breast, weakly in the normal sample and strongly in the matched tumour sample. This provided further evidence that this novel gene could be a very powerful molecular marker for the presence of a breast tumour.

DD53 was further tested using cDNA populations derived from a panel of 22 human tissue types by real-time PCR analysis. In addition, assays were performed on a range of ethically approved human tumour samples, obtained through Medical Solutions plc, to ascertain whether the marker was breast tumour specific or a less specific marker for the presence of cancer. cDNA representative of tumours from ovary, testis, colon, stomach, liver, lung, bladder and pancreas were also tested. DD53 was evident in most of the cDNA samples at low levels, however, levels of expression varied, with this marker reaching the threshold of detection in several cDNA populations many cycles earlier than in others. This result was confirmed by conventional PCR, using the same templates. DD53 was detected early, indicative of a higher starting population, in cDNA derived from placenta, prostate, testis, uterus, bladder tumour and ovarian tumour. The results from this comprehensive panel appear to be at odds with those from the Origene panel, but the substantially increased expression of this marker in the samples listed above indicate that diagnosis may be determined by its increased expression, rather than presence or absence. Differences between the Origene and total human panel may be due to the inherent variability of the source tissue samples, as 38 cycles of amplification were used for both PCR sets.

Using combined data from the molecular signature profiles of all matched tissue samples, cluster analysis was performed on the expression profiles of the molecular markers used, including DD53. Despite a small matched sample number, the DD53 expression profile clustered very strongly with ERα, progesterone receptor (PR) and Bcl-1, suggesting that this novel marker would be an independent indicator of ERα-positive breast tumours, facilitating sub-division of cancers of this type. As a further reference, ERα was tested against the human cDNA panel and the expression profile was similar to that for DD53, with higher expression noted in prostate, testis, uterus and ovarian tumour samples (data not shown).

In conclusion, the close similarities in the expression of DD53 to ERα, and its elevated levels in a small number of human tissues indicate that this molecular marker may have utility in the diagnosis of breast cancer and more specifically for the sub-grouping of this disease. Despite this marker being expressed in many tissue samples, elevated expression is mainly limited to organs under the influence of sex hormones, such as testis and ovary, so DD53 may also be of value in other cancer diagnostics.

### TS32

Using differential display, a gene fragment, designated TS32, derived from cDNA populations of matched tissue from a breast cancer donor was observed to have significant up-regulation in the tumour cDNA population in comparison to the corresponding normal tissue cDNA. This 232-nucleotide product; 221 nucleotides without the poly A tail (SEQ ID No 10) was confirmed as differentially expressed by reverse Southern dot blots. Sequence analysis followed by database interrogation determined that TS32 was not homologous to known genes or proteins in the EMBL and SWISSPROT databases, respectively, so was regarded as potentially novel. It was, however, 100% homologous, to a predicted gene (rorchee), on chromosome 11p11, predicted by Acembly Gene Predictions, sourced through a BLAST search of the human genome. The predicted gene comprises 621 nucleotides (SEQ ID No 11) and contains a presumed ORF of 56 amino acids (SEQ ID No 12).

The TS32 fragment was screened using cDNA populations derived from a number of matched breast tumour tissues donated by other cancer patients. Of the donor samples screened, 14 out of 19 exhibited notable increases in expression, as shown in the table below, confirming TS32 to be a putative molecular marker for the presence of breast tumour.

| | | |
|---|---|---|
| TS32 Increased in tumour | 14 | 73.7% |
| TS32 Increased in normal | 3 | 15.7% |
| TS32 No discernable difference | 1 | 5.3% |
| TS32 No expression evident | 1 | 5.3% |
| Totals | 19 | 100% |

To compare the expression of the predicted gene homologue against the original TS32 fragment, ORF primers were designed for rorchee and used against the matched breast cancer panel; the expression profile of the product derived from the ORF primer set against this tissue panel was found to be the same, so the rorchee gene is considered to be the full-length equivalent of TS32. This molecular marker did not show increased expression in all tumour samples from the matched sets, so may be a useful tool for the sub-classification of breast cancer, either in isolation or as part of an array of marker genes.

TS32 was further tested using cDNA populations derived from a panel of 22 human tissue types by real-time PCR analysis. Of those cDNA populations tested, TS32 was detected in most tissues at low levels (data not shown), indicating that the marker is not tissue specific. In addition, assays were performed on a range of ethically approved human tumour samples, obtained through Medical Solutions, to ascertain whether the marker was breast tumour specific or a less specific tumour marker. TS32 was again present in most tumours at low levels (data not shown), so cannot be regarded as a specific marker for breast cancer. This may therefore have utility as a general indicator for the presence of a tumour, through elevated expression, rather than simply presence or absence.

On the basis of the present results, TS32 can be considered a very strong indicator of cancer in the context of breast specific assays, using biopsy samples, for example. In addition, its lack of uniformity among the matched samples tested may indicate a role in the sub-division of breast tumour types or stages. Higher volume screening is underway to ascertain whether this promising marker can be associated with a particular sub-group of breast cancer or whether it can be used as a marker for other cancer types in addition to breast cancer.

### Results: IDC25

Using differential display, a gene fragment, IDC25, derived from cDNA populations of matched tissue from 2 invasive ductal carcinoma (IDC) donors, was observed to have significant up-regulation in both the tumour cDNA populations in comparison to their corresponding normal tissue cDNA. This 367-nucleotide product; 356 nucleotides without poly A tail (SEQ ID No. 13) was confirmed as differentially expressed by reverse Southern dot blots. Sequence analysis followed by database interrogation determined that IDC25 was 100% homologous over its complete sequence to S19, a ribosomal protein found on chromosome 19q of the human genome. This ribosomal protein has an overall length of 873 nucleotides (SEQ ID No 14), a presumed open reading frame of 438 nucleotides (145 amino acids, SEQ ID No 15) and was first identified by Strausberg et al. (2002), as one of several thousand human genes cloned as part of the mammalian gene collection programme. At the genomic level, the complete S19 gene is interrupted by 5 introns, with the IDC25 fragment having 3 of these, stopping just short of the full ORF.

A detailed real-time expression profile of this fragment was undertaken using cDNA populations derived from a number of matched breast tumour tissues donated by other patients. Of the samples screened, a number exhibited notable increases in expression in the tumour cDNA populations, confirming IDC25 to be a putative molecular marker for the presence of breast cancer (FIG. 18 shows 12 of these matched sets). The screening of all other available matched breast tissue samples substantiated this analysis suggestion, as follows;

| | | |
|---|---|---|
| IDC25 Increased in tumour | 10 | 62.5% |
| IDC25 Increased in normal | 1 | 6.2% |
| IDC25 No discernable difference | 5 | 31.3% |
| IDC25 No expression evident | 0 | 00.0% |
| Totals | 16 | 100% |

To facilitate further analysis, the presumed open reading frame of the gene homologue (S19) was used for the design of internal ORF primers. Expression profiling of this marker was repeated, using the ORF primer set, with comparable results to the initial IDC25 fragment profile (data not shown). To determine tissue specificity, this molecular marker was further tested using cDNA populations derived from a panel of 22 human tissue types by real-time PCR analysis. Of those tested, IDC25 was detected in all samples from the panel (data not shown). In addition, assays were performed on a range of ethically approved human tumour samples, obtained through Medical Solutions plc, to ascertain whether the marker was breast tumour specific or a less specific marker for the presence of tumour in general. cDNA representative of tumours from ovary, testis, colon, stomach, liver, lung, bladder and pancreas was tested, with IDC25 being detected in all cases (data not shown).

Despite this marker being present in all human tissue and tumour samples tested, the increase in expression of IDC25 in over 60% of the breast tumour populations indicates that this marker has utility as an indicator for the presence of breast cancer. The variability of detection of IDC25 may also enable its use as a classifier of specific breast tumour sub-types or stages and high volume screening is underway to determine if this is the case. Molecular signature data from all tissues sampled is also being analysed to determine any associations between this marker and pre-published cancer markers. In addition, its presence in other tumours may indicate a potential role in the diagnosis of other cancers, detectable through elevation of expression.

### REFERENCES

1. DeRisi, J. L., Iyer, V. R. and Brown, P. O. 1997. Exploring the metabolic and genetic control of gene expression on a genomic scale. Science. 278: 680-686.
2. Grover, P. L. and Martin, F. L. 2002. The initiation of breast and prostate cancer. Carcinogenesis. 23 (7): 1095-1102.
3. Hames, B. D. and Higgins, S. J., (Editors). 1985. Nucleic Acid Hybridisation. A Practical Approach. IRL Press.
4. Keen, J. C. and Davidson, N. E. 2003. The biology of breast carcinoma. Cancer. 97 (3-Supplement): 825-833.
5. Liang, P. and Pardee, A. B. 1992. Differential display of eukaryotic messenger RNA by means of the polymerase reaction. Science. 257: 967-971.
6. Ma, Xiao-Jun., Ranelle Salunga, J. Todd Tuggle, Justin Gaudet, Edward Enright, Philip McQuary, Terry Payette, Maria Pistone, Kimberly Stecker, Brian M. Zhang, Yi-Xiong Zhou, HeikeVarnholt, Barbara Smith, Michelle Gadd, Erica Chatfield, Jessica Kessler, Thomas M. Baer, Mark G. Erlander, and Dennis C. Sgroi. 2003. Gene expression profiles of human breast cancer progression. Proc Natl Acad Sci USA. 100 (10): 5974-5979.
7. Pritzker, K. P. 2002 Cancer biomarkers: easier said than done. Clin. Chem. 2002 August; 48(8):1147-50.
8. Salodof MacNeil. 2001. From genes to proteins: The FLEXgene consortium. HMS Beagle. 112: on-line journal.
9. Sorlie T, Perou C M, Tibshirani R, Aas T, Geisler S, Johnsen H, Hastie T, Eisen M B, van de Rijn M, Jeffrey S S, Thorsen T, Quist H, Matese J C, Brown P O,
10. Botstein D, Eystein Lonning P, Borresen-Dale A L. 2001. Gene expression patterns of breast carcinomas distinguish tumor subclasses with clinical implications. Proc Natl Acad Sci USA. September 11; 98(19):10869-74.
11. Srinivas P R, Verma M, Zhao Y, Srivastava S. 2002. Proteomics for cancer biomarker discovery. Clin Chem. August; 48(8):1160-9.
12. Strausberg R. L., Feingold E. A., Grouse L. H., Derge J. G., Klausner R. D. 2002. Generation and initial analysis of more than 15,000 full-length human and mouse cDNA sequences. Proc. Natl. Acad. Sci. U.S.A. 99(26): 16899-16903.
13. Sturtevant, J. Applications of differential-display reverse transcription-PCR to molecular pathogenesis and medical mycology. Clin Microbiol Rev. 2000 July; 13(3):408-27.
14. Yousef et al., 2002 Yousef G M, Scorilas A, Kyriakopoulou L G, Rendl L, Diamandis M, Ponzone R, Biglia N, Giai M, Roagna R, Sismondi P, Diamandis E P. Human kallikrein gene 5 (KLK5) expression by quantitative PCR: an independent indicator of poor prognosis in breast cancer. Clin Chem. 2002 August; 48(8):1241-50.
15. Zong, Q., Schummer, M., Hood, L. and Morris, D. R. 1999. Messenger RNA translation state: the second dimension of high-throughput expression screening. Proc. Natl. Acad. Sci. 96: 10632-10636.

1. A method for the detection of the presence of or the risk of cancer in a patient comprising the steps of:
(i) isolating a sample of the patient's genome; and
(ii) detecting the presence or expression of the gene characterised by any of the nucleotide sequences identified as SEQ ID No. 1, SEQ ID No. 10 and SEQ ID No. 13, wherein the presence or expression of the gene indicates the presence of or the risk of cancer.

2. A method according to claim 1, wherein the sample is obtained from breast tissue.

3. A method according to claim 1 wherein the cancer is breast cancer.

4. A method according to claim 1, wherein detection is carried out by amplifying the gene using the polymerase enzyme.

5. An isolated polynucleotide comprising the nucleotide sequence identified herein as SEQ ID. No. 1, 10 or 13, or its complement, or a polynucleotide of at least 15 consecutive nucleotides that hybridises to the sequence (or its complement) under stringent hybridising conditions.

6. An in vitro diagnostic assay to test for the presence of or the risk of cancer in a patient, comprising testing a biological sample from said patient for the presence of a polynucleotide comprising the nucleotide sequence identified herein as SEQ ID. No. 1, 10 or 13, or its complement, or a polynucleotide of at least 15 consecutive nucleotides that hybridises to the sequence (or its complement) under stringent hybridising conditions, wherein the presence of such a polynucleotide indicates that said patient has cancer or is at risk of having cancer.

7. The method according to claim 6, wherein the cancer is breast cancer.

8. A peptide comprising any of the sequences identified herein as SEQ ID Nos. 9, 12 or 15 or a fragment thereof of at least 10 consecutive amino acid residues.

9. An antibody having affinity of at least 10⁶M for the peptide of claim 9.

10. A method for treating cancer in a subject comprising administering to said subject a polynucleotide that hybridises with or inhibits the expression of an endogenous gene that comprises the polynucleotide a polynucleotide sequence identified herein as SEQ ID. No. 1, 10 or 13 in an amount affective to alleviate one or more symptoms of said cancer.

11. A method according to claim 2, wherein the cancer is breast cancer.

12. A method according to claim 2, wherein detection is carried out by amplifying the gene using the polymerase enzyme.

13. A method according to claim 3, wherein detection is carried out by amplifying the gene using the polymerase enzyme.

14. A method according to claim 10, wherein the cancer is breast cancer.
